(19) United States
(12) Patent Application Publication
(10) Pub. No.: US 2013/0209418 A1
(43) Pub. Date: Aug. 15, 2013

(54) METHODS AND COMPOSITION RELATED TO BROWN ADIPOSE-LIKE CELLS

(71) Applicant: JOHNSON & JOHNSON REGENERATIVE THERAPEUTICS, LLC, (US)

(72) Inventors: Agnieszka Seyda, Belle Mead, NJ (US); David C. Colter, Hamilton, NJ (US); Charito S. Buensuceso, North Brunswick, NJ (US); Christian C. Kazanecki, Martins Creek, PA (US); Sridevi Dhanaraj, Raritan, NJ (US)

(73) Assignee: JOHNSON & JOHNSON REGENERATIVE THERAPEUTICS, LLC, Raynham, MA (US)

(21) Appl. No.: 13/765,008

(22) Filed: Feb. 12, 2013

Related U.S. Application Data
(60) Provisional application No. 61/599,080, filed on Feb. 15, 2012.

(51) Int. Cl. C12N 5/071; A61K 35/12

(52) U.S. Cl. CPC C12N 5/0653; A61K 35/35
USPC 424/93.7; 435/325; 435/377

(57) ABSTRACT

Methods and therapeutics are provided for treating diseases, including metabolic diseases and other weight-related disorders. Generally, methods for making brown adipose-like including culturing a population of artery-derived cells in adipogenic induction medium for a period of time and under conditions sufficient to increase expression of at least one adipocyte marker at a higher level as compared to untreated artery-derived cells are disclosed. Isolated artery-derived, ex vivo differentiated brown adipose-like cells are also provided, including pharmaceutical compositions and cell delivery systems thereof. In another embodiment, a method of treating a subject is disclosed that includes obtaining a population of artery-derived brown adipose-like cells and administering the brown adipose-like cells into a target region in the subject.

### CROSS REFERENCE TO RELATED APPLICATIONS

The present application claims the priority of U.S. Provisional Application Ser. No. 61/599,080 filed Feb. 15, 2012, entitled “Methods and Composition Related to Brown Adipose-Like Cells,” which is hereby incorporated by reference in its entirety.

### FIELD OF THE INVENTION

The present invention relates to methods and compositions related to brown adipose-like cells and the treatment of metabolic disease and other diseases.

### BACKGROUND OF THE INVENTION

Obesity represents the most prevalent of body weight disorders, and it is the most important nutritional disorder in the Western world, with estimates of its prevalence ranging from 30% to 50% of the middle-aged population. The number of overweight and obese Americans has continued to increase since 1960, a trend that is not slowing down. Today, approximately 64.5 percent of adult Americans are categorized as being overweight or obese. Obesity is becoming a growing concern as the number of people with obesity continues to increase and more is learned about the negative health effects of obesity. Each year, obesity causes at least 300,000 deaths in the U.S., and healthcare costs of American adults with obesity amount to more than $125 billion (American Obesity Association). Severe obesity, in which a person is 100 pounds or more over ideal body weight, in particular poses significant risks for severe health problems. Accordingly, a great deal of attention is being focused on treating patients with obesity.

Even mild obesity increases the risk for premature death, diabetes, hypertension, atherosclerosis, gallbladder disease and certain types of cancer. Because of its high prevalence and significant health consequences, its treatment should be a high public health priority. Therefore, a need exists for better methods and therapeutics for treating obesity and inducing weight loss.

### SUMMARY OF THE INVENTION

The present invention generally provides methods and compositions for treating diseases, including metabolic diseases and weight-related disorders involving increasing brown adipose tissue, supplementing brown adipose tissue or replacing white adipose tissue with brown adipose-like tissue. One aspect discloses methods and compositions for isolated artery-derived, ex vivo differentiated brown adipose-like cells. Another aspect discloses methods and compositions for treating a subject by obtaining a population of artery-derived brown adipose-like cells and administering the brown adipose-like cells into a target region in the subject.

In another embodiment, the method of making brown adipose-like cells can include increasing expression of an adipocyte marker selected from fatty acid binding protein 4 (aP2), peroxisome proliferator activated receptor α (PPARα) peroxisome proliferator activated receptor γ (PPARγ), adiponectin (ADN or ADIPOQ), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3). Furthermore, the adipocyte marker can be a brown adipocyte marker, such as uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3). The method can further include isolating the brown adipose-like cells.

Furthermore, the artery-derived cells can be internal mammary artery cells or iMACs. These cells can be positive for HLA-1 and negative for CD10, CD31, CD34, CD45, CD133, CD141, and KDR/Flk-1. The artery-derived cells can be additionally positive for CD29, CD44, CD73, CD166, and additionally negative for CD15, CD23, CD24, CD62p, CD80, CD86, CD104, CD117, CD138, CD146, VE-Cadherin, and HLA-2.

The method of making brown adipose-like cells includes culturing the population of artery-derived cells in adipogenic induction medium. The adipogenic induction medium can include a compound or a combination of compounds selected from bone morphogenetic proteins (BMP), peroxisome proliferator-activated receptor gamma (PPARγ), Retinoid X receptor-alpha (RxRα), insulin and T3, a thiazolidinedione (TZD), vitamin A, retinoic acid, insulin, glucocorticoid or agonist thereof, Wingless-type (Wnt), Insulin-like Growth Factor-1 (IGF-1), Epidermal growth factor (EGF), Fibroblast growth factor (FGF), Transforming growth factor (TGF)-α, TGF-β, Tumor necrosis factor alpha (TNFα), Macrophage colony stimulating factor (MCSF), Vascular endothelial growth factor (VEGF) and Platelet-derived growth factor (PDGF).

An exemplary embodiment includes a population of cells made by the disclosed method of culturing a population of artery-derived cells in adipogenic induction medium for a period of time and under conditions sufficient to increase expression of at least one adipocyte marker at a higher level as compared to untreated artery-derived cells.

Another aspect includes isolated artery-derived, ex vivo differentiated brown adipose-like cells. The brown adipose-like cells can be characterized by expression of at least one adipocyte marker selected from fatty acid binding protein 4 (aP2), peroxisome proliferator activated receptor α (PPARα) peroxisome proliferator activated receptor γ (PPARγ), adiponectin (ADN or ADIPOQ), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3). Additionally, the adipocyte marker can be a brown adipocyte marker selected from uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3). The adipogenic marker can be expressed in the brown adipose-like cell at higher levels as compared to untreated artery-derived cells.

The brown adipose-like cells can further be characterized by their thermogenic potential. This specialized function of brown adipose cells derives from high mitochondrial content and the ability to uncouple cellular respiration causing proton leak across the mitochondrial membrane through physical or chemical stimulation or signaling through upstream receptors of uncoupling protein (UCP) to generate heat. The thermogenic potential can be stimulated by exposure to at least one of catecholamine and cyclic AMP.

Moreover, the isolated artery-derived, ex vivo differentiated brown adipose-like cells can be differentiated from internal mammary artery cells or iMACs.

The isolated artery-derived, ex vivo differentiated brown adipose-like cells can also be included in a pharmaceutical composition with a pharmaceutically acceptable carrier. Alternatively, the isolated artery-derived, ex vivo differentiated brown adipose-like cells can be included in a cell delivery system with a reservoir containing the brown adipose-like cells in a pharmaceutically acceptable carrier, and a delivery device in fluid contact with the reservoir. In an exemplary embodiment, the reservoir can be a needle or cannula. Moreover, the delivery device can house the brown adipose-like cells in a single container or chamber of a housing, such as a vial or syringe.

In another aspect, a method of treating a subject by obtaining a population of artery-derived brown adipose-like cells and administering the brown adipose-like cells into a target region in the subject is disclosed. In one embodiment, the method further includes preparing the brown adipose-like cells as an injectable composition.

In another embodiment, the artery-derived brown adipose-like cells can be autologous to the subject. Alternatively, the brown adipose-like cells can be allogeneic, or xenogeneic to the subject.

The subject can also have a metabolic disorder selected from obesity, diabetes or hyperlipidemia. Additionally, the subject can be obese and in need of treatment. In an exemplary embodiment, the subject is human.

The method of treating a subject can also increase thermogenic potential in the subject. Thermogenic potential can be characterized as proton leak across the mitochondrial membrane that generates heat. Additionally, the method can include stimulating the artery-derived brown adipose-like cells to increase thermogenic potential to treat the subject.

### BRIEF DESCRIPTION OF THE DRAWINGS

The invention will be more fully understood from the following detailed description taken in conjunction with the accompanying drawings, in which:

FIG. 1 is an illustration of mesenchymal stem cell differentiation into white adipocytes, brown adipocytes, myocytes and osteocytes;

FIG. 2A shows internal mammary artery cells (iMACs) exposed to adipogenic medium and stained with Oil Red O solution had a marked increase in lipid accumulation;

FIG. 2B shows iMACs exposed to control medium (maintenance medium) and stained in Oil Red O solution displayed no significant lipid accumulation;

FIG. 3A shows relative quantitative RT-PCR expression levels of adipocyte markers fatty acid binding protein 4 (aP2), peroxisome proliferator activated receptor α (PPARα) peroxisome proliferator activated receptor γ (PPARγ), adiponectin (ADN or ADIPOQ) in differentiated iMACs as compared to untreated iMACs; and

FIG. 3B shows relative quantitative RT-PCR expression levels of brown adipocyte markers uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3) in differentiated iMACs as compared to untreated iMACs.

### DETAILED DESCRIPTION OF THE INVENTION

Certain exemplary embodiments will now be described to provide an overall understanding of the principles of the structure, function, manufacture, and use of the therapeutics and methods disclosed herein. One or more examples of these embodiments are illustrated in the accompanying drawings. Those skilled in the art will understand that the therapeutics and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present disclosure provides compositions and methods useful for increasing brown adipose tissue (BAT) and/or BAT function in a subject for treating diseases, such as obesity and other weight related diseases and disorders. These methods include promoting the differentiation of progenitor cells (e.g., progenitor cells capable of differentiating into adipose cells) to or towards a BAT cell lineage. More specifically, the present disclosure is based, at least in part, on the discovery that artery-derived cells, such as internal mammary artery cells, are capable of differentiating to or towards brown adipose-like cells or BAT cell lineages. As described herein, these compositions and methods can be used to increase the BAT cell number and/or BAT function and/or to increase the ratio of BAT to white adipose tissue (WAT) and thereby treat metabolic diseases such as obesity and weight related diseases and disorders in a subject.

Some of the methods described herein include implanting artery-derived cells that have been treated with adipogenic induction medium. In general, the methods include treating (e.g., contacting) progenitor cells, e.g., artery-derived cells, with the adipogenic induction or differentiation medium, and thereafter implanting the adipose-like cells (e.g., at least one cell or a population of such cells) in a subject.

### Brown Adipose Tissue

Body mass index (BMI) is a measure expressing the relationship (or ratio) of weight-to-height based on a mathematical formula in which a person's body weight in kilograms is divided by the square of his or her height in meters (i.e., wt/(ht)²). See National Institute of Health, Clinical Guidelines on the Identification, Evaluation, and Treatment of Overweight and Obesity in Adults (1998).

Obesity typically refers to an individual having a BMI of 30 kg/m² or more. Overweight describes an individual having a BMI of 25 kg/m² or greater, but less than 30 kg/m². However, not all individuals with metabolic diseases are obese or overweight. Non-obese individuals may have metabolic diseases, such as diabetes and hyperlipidemia, with BMI of less than 25 kg/m².

Adipocytes are central to the control of energy balance and lipid homeostasis. The ability to store excess energy in adipose tissue is an important evolutionary adaptation. There are two types of fat or adipose tissue: white adipose tissue (WAT), the primary site of energy storage, and brown adipose tissue (BAT), specialized for energy expenditure and thermogenesis. Intriguingly, an inverse correlation exists between the amount of brown adipose tissue and body mass index, with obese individuals having significantly less of the tissue than lean individuals; this suggests that brown fat may be an important factor in maintaining a lean phenotype or that the obese phenotype has led to the diminution in size and/or activity of the BAT depots.

Adipose tissue is composed, in part, of adipocytes or adipose cells specific for WAT or BAT. Adipocytes can also produce adipokines, such as tumor necrosis factor α (TNFα), leptin, resistin, retinol binding protein 4 (RBP4), apelin, and adiponectin, to modulate systemic metabolism. The inability to properly store triglycerides in adipose tissue results in adverse effects on glucose metabolism in the liver and skeletal muscle. In contrast with WAT, the physiological role of BAT is to metabolize fatty acids and expend energy through thermogenesis. This specialized activation function of brown adipose cells derives from high mitochondrial content and the ability to uncouple cellular respiration through physical or chemical stimulation or signaling through upstream receptors of uncoupling protein (UCP) to generate heat. Thermogenesis is the heat production caused by the metabolic rate activated by exposure to cold. For example, brown adipose cells become activated and exhibit thermogenic potential due to proton leak across the mitochondrial membrane that generates heat. This functional potential can also be stimulated by exposure to at least one of a catecholamine, like norepinephrine, cyclic AMP and leptin. Due to these functional differences between WAT and BAT, the ratio of WAT to BAT can affect systemic energy balance that may contribute to the development of obesity.

Methods and compositions are disclosed to increase BAT activity or energy expenditure by increasing the total amount of BAT in a subject. This can be achieved through multiple mechanisms, such as differentiation of stem/progenitor cells to brown adipose cells, e.g. inducing differentiation of artery-derived cells into brown adipose-like cells; and transplantation of stem/progenitor cells, induced pluripotent stem cells (iPSC), artery-derived cells, brown adipose cells, and/or brown adipose-like cells into BAT depots or any other site with sufficient innervations and vascularity.

Differentiation into Brown Adipose-Like Cells

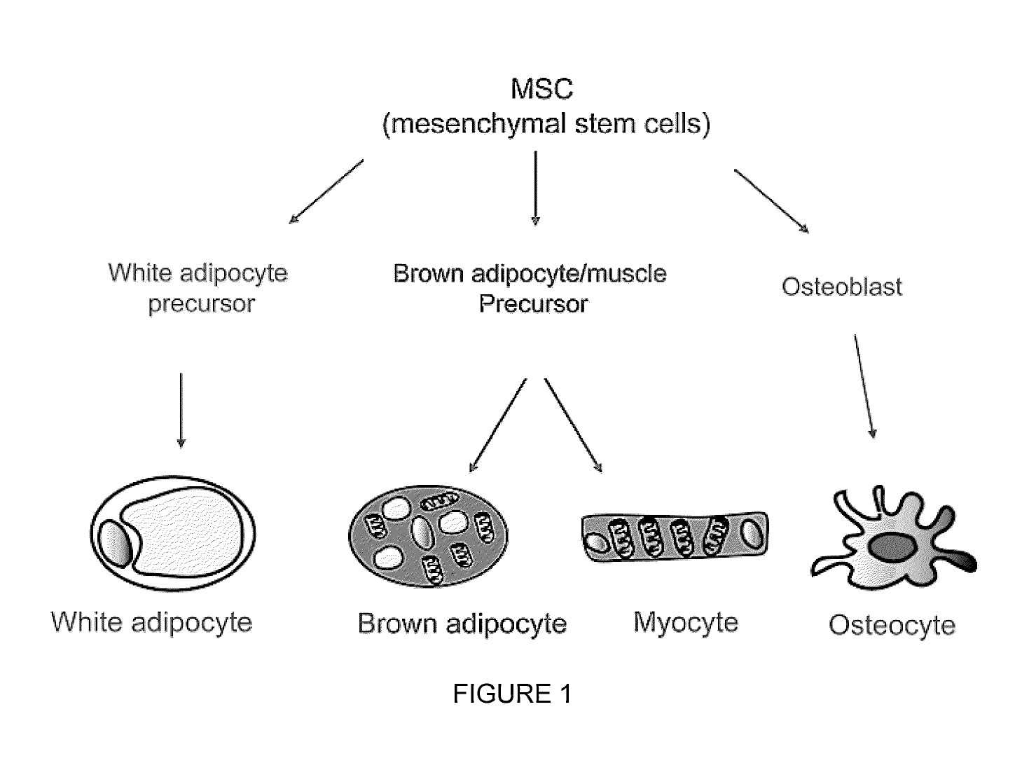

Mesenchymal stem cells give rise to precursor cells of bone, muscle, and fat cells under appropriate conditions. See FIG. 1. Generally, brown fat cells come from the middle embryo layer, or mesoderm, the source of myocytes (muscle cells), adipocytes, and chondrocytes (cartilage cells). Adipogenesis is generally described as a two-step process. The first step comprises the generation of committed adipocyte progenitors (or preadipocytes) from mesenchymal stem cells (MSCs). The second step involves the terminal differentiation of these preadipocytes into mature functional adipocytes.

As used herein, the terms “adipocyte” or “adipose cell” encompass both white adipose cells and brown adipose cells. The terms “brown adipocyte,” and “brown adipose cell” are used interchangeably. The terms “artery-derived cell,” and “adipocyte precursor cell,” as used herein, refer to a cell that can proliferate and be induced to differentiate to a brown adipose-like cell. The adipocyte precursor cell encompasses, but is not limited to, an artery-derived cell, such as an internal mammary artery cell (iMAC), and other cells that can be differentiated to produce brown adipose-like cells. The terms “proliferate,” “proliferation” or “proliferated” may also be used interchangeably with the words “expand,” “expansion,” or “expanded.”

Augmenting the number of BAT cells to increase overall energy expenditure in a subject can provide a mechanism to treat metabolic disorders, such as obesity, diabetes and hyperlipidemia. In one embodiment, brown adipose tissue can be augmented by isolation of adipocyte precursor cells, differentiation of the adipocyte precursor cells into brown adipose-like cells and transfer of the brown adipose-like cells into brown adipose tissue.

In one aspect, adipocyte precursor cells, such as artery-derived cells, can be removed from the body and cultured to grow and expand. Artery-derived cells, such as internal mammary cells (iMACs), can be cultured in any appropriate medium that maintains the viability and proliferative state of the cells, such as a growth medium. For example, iMACs isolated from mammalian internal mammary arteries are self-renewing and can be differentiated into brown adipose-like cells, in addition to producing daughter cells of equivalent potential. These cells are “isolated” from the internal mammary artery, which refers to the separation of the cells from the surrounding tissue as disclosed in U.S. Appl. Pub. No.: 2011/0076769. The term “isolated” as used herein refers to a cell, a group of cells, a population of cells, a tissue or an organ that has been purified from the other components.

Conventional methods techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required. Some non-limiting examples can include, but are not limited to, characterizing adiopocyte precursor cells by their transcriptome, cytokine profile, proteome, and cell surface biomarker detection. The terms “biomarker,” “surface marker,” and “marker” are used interchangeably and refer to a protein, glycoprotein, receptor or other molecule expressed on the surface of a cell, which serves to help identify the cell. The cell surface markers can generally be detected by conventional methods known by those skilled in the art. Specific, non-limiting examples of methods for detection of a cell surface marker are immunohistochemistry, fluorescence activated cell sorting (FACS), or an enzymatic analysis.

Adipocyte precursor cells can be identified through expression of one or more markers of interest to bind to the solid-phase linked antibodies. The bound cells are then separated from the solid phase by any appropriate method, depending mainly upon the nature of the solid phase and the antibody employed. Antibodies may be conjugated to biotin, which then can be removed with avidin or streptavidin bound to a support or fluorochromes, which can be used with a fluorescence activated cell sorter (FACS), to enable cell separation.

In one embodiment, adipocyte precursor cells, such as iMACs, can be characterized by positive expression of HLA-1 and negative expression of CD10, CD31, CD34, CD45, CD133, CD141, and KDR/Flk-1. iMACs can also be characterized by being additionally positive for CD29, CD44, CD73, CD166, and additionally negative for CD15, CD23, CD24, CD62p, CD80, CD86, CD104, CD117, CD138, CD146, VE-Cadherin, and HLA-2. While early experiments showed iMACs to be negative for CD105, further experiments showed iMACs to be positive for CD105.

Artery-derived cells can be isolated from arterial tissues, such as the internal mammary artery. Cells can be isolated by a variety of methods, including mechanical and/or enzymatic methods. In one embodiment, an isolated population of cells includes greater than about 50%, greater than about 70%, greater than about 75%, greater than about 80%, greater than about 85%, greater than about 90%, greater than about 95%, greater than about 96%, greater than about 97%, greater than about 98%, or greater than about 99% of the cells of interest. The cells of interest can include, but is not limited to, adipocyte precursor cells, adipocytes, and brown adipose-like cells. In another embodiment, an isolated population of cells is one in which other cells of a phenotype different than the cells of interest cannot be detected. In a further embodiment, an isolated population of cells is a population of cells that includes less than about 15%, less than about 10% of cells, less than about 5% of cells, less than about 4% of cells, less than about 3% of cells, less than about 2% of cells or less than about 1% of cells of a different phenotype than the cells of interest.

Separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well within the skill in the art.

In some methods, a subpopulation of cells can be isolated according to adherence to a solid substrate (referred to as “adherent cells”), such as a cell culture container (for example, a culture dish, a culture flask, or beads designed for tissue culture). In some embodiments the solid substrate can comprise an extracellular matrix (ECM) substrate. ECM substrates include, for example, fibronectin, collagen, laminin, vitronectin, polylysine, tenascin, elastin, proteoglycans (such as, heparan sulfate proteoglycans), entactin, Matrigel™, synthetic RGDS-containing peptides covalently crosslinked to hydrophobic biocompatible scaffolds (such as polyethylene glycol (PEG), poly glycolic acid (PGA), poly(D,L-lactide-co-glycolide) (PLGA), or others), or a combination thereof. Any or all forms of a particular ECM substrate are contemplated herein. For example, collagen is commonly known to occur in multiple isoforms (Molecular Biology of the Cell, 3rd Edition, ed. by Alberts et al., New York: Garland Publishing, 1994, Ch. 19), including eighteen different collagen isoforms (such as collagen I, II, III, IV, V, and others). Similarly, multiple isoforms of laminin (Ekblom et al., Ann. N.Y. Acad. Sci., 857:194-211, 1998) and fibronectin ((Molecular Biology of the Cell, 3rd Edition, ed. by Alberts et al., New York: Garland Publishing, 1994, Ch 19) are known. In specific, non-limiting embodiments, an ECM substrate comprises a 1-1000 ng/ml fibronectin-coated solid substrate, for example a 10 ng/ml fibronectin-coated solid substrate.

Numerous culture media are known and are suitable for maintaining adipocyte precursor cells in culture. Generally, a growth medium includes a minimal essential medium. In one embodiment, the medium is DMEM/F12. The growth medium may be supplemented with serum. Specific, non-limiting examples of serum are horse, calf or fetal bovine serum (FBS). The medium can have between about 2% by volume to about 20% by volume serum, or about 5% by volume serum, or about 10%. In one embodiment, a growth medium is supplemented with about 10% FBS. In one embodiment, the medium contains one or more additional additives, such as antibiotics or nutrients. Nutrients can include amino acids, such as 10-1000 U/ml L-glutamine. Specific non-limiting examples of antibiotics include 10-1000 U/ml penicillin and about 0.01 mg/ml to about 10 mg/ml streptomycin. In a particular example, a growth medium contains about 50 U/ml L-glutamine, 50 U/ml penicillin and about 50 μg/ml streptomycin.

The culture medium can be any medium or any buffer that maintains the viability of the cells, such as a growth medium. Numerous culture media are known and are suitable for use. Generally, a growth medium includes a minimal essential medium. In one embodiment, the medium is DMEM-low glucose (DMEM-LG). The growth medium may be supplemented with serum. Specific, non-limiting examples of serum are horse, calf or fetal bovine serum (FBS). The medium can have between about 2% by volume to about 10% by volume serum, or about 5% by volume serum, or about 2%. In one embodiment, a growth medium is supplemented with about 5% FBS. In one embodiment, the medium contains one or more additional additives, such as antibiotics or nutrients. Specific non-limiting examples of antibiotics include 10-1000 U/ml penicillin and about 0.01 mg/ml to about 10 mg/ml streptomycin. In a particular example, a growth medium contains about 100 U/ml penicillin and about 1 mg/ml streptomycin.

The adipocyte precursor cells, such as artery-derived cells or iMACs, can be expanded in growth medium. Single-cell-derived colonies of adipocyte precursor cells, such as artery-derived cells or iMACs, may be isolated for expansion using any technique known in the art, such as cloning rings. Alternatively, single-cell-derived colonies of adipocyte precursor cells, such as artery-derived cells or iMACs, may be pooled for expansion. In a particular embodiment, the iMACs are cultured in growth medium for a sufficient number of days to obtain a desired number of iMACs. In one embodiment, the iMACs are cultured in growth medium for at least about 2 day. In another embodiment, the iMACs are cultured in growth medium for at least about 7 days. In yet another embodiment, the iMACs are cultured in growth medium for at least about 14 days.

In another aspect, brown adipose-like cells can be generated through differentiation of adipocyte precursor cells, such as artery-derived cells or iMACs. Adipocyte precursor cells, such as artery-derived cells or iMACs, can be induced differentiate into brown adipose-like cells useful with the present disclosure. The terms “differentiate” and “differentiation” as used herein refer to a process whereby relatively unspecialized cells (for example, undifferentiated cells, such as multilineage-inducible cells) acquire specialized structural and/or functional features characteristic of mature cells. Typically, during differentiation, cellular structure alters and tissue-specific proteins appear. “Adipogenic differentiation” is a process whereby undifferentiated cells acquire one or more properties (for example, morphological, biochemical, or functional properties) characteristic of adipocytes, e.g., brown adipocytes. One skilled in the art will appreciate that the “brown adipose-like cells” include brown adipocytes that derive from MSCs, adipocyte progenitor cells, pre-adipocytes and artery-derived cells.

Induction of differentiation of adipocyte precursor cells to brown adipose-like cells can be performed by methods known by those skilled in the art. For example, known methods can include, but are not limited to, treatment of adipocyte precursor cells with compounds such as ligands for nuclear hormone receptors (dexamethasone) and peroxisome proliferator-activated receptor γ (PPAR γ, pioglitazone, rosiglitazone, Avandia™), indomethacin, insulin, thiazolidinedione, and compounds that increase intracellular levels of cAMP (isobutylmethylxanthine). In one embodiment, adipocyte precursor cells are cultured in adipogenic induction medium that includes one or more of hydrocortisone, ligands for nuclear hormone receptors (dexamethasone) and peroxisome proliferator-activated receptor γ (PPAR γ, pioglitazone, rosiglitazone, Avandia™), bone morphogenetic proteins (BMP), Retinoid X receptor-alpha (RxRα), insulin and T3, a thiazolidinedione (TZD), vitamin A, retinoic acid, insulin, glucocorticoid or agonist thereof, Wingless-type (Wnt), Insulin-like Growth Factor-1 (IGF-1), Epidermal growth factor (EGF), Fibroblast growth factor (FGF), Transforming growth factor (TGF)-α, TGF-β, Tumor necrosis factor alpha (TNFα), Macrophage colony stimulating factor (MCSF), Vascular endothelial growth factor (VEGF) and Platelet-derived growth factor (PDGF), indomethacin, and compounds that increase intracellular levels of cAMP (isobutylmethylxanthine). Adipocyte precursor cells can also be induced to differentiate through expression or overexpression of molecules known to induce differentiation. These can include, but are not limited to, treatment with bone morphogenic proteins, e.g., BMP7, PPARγ, myogenic factor 5 (myf5), PR domain containing 16 (PRDM16), and transfection of transcriptional regulators such as PRDM16 and PPARγ to induce differentiation.

In an exemplary example, the adipogenic induction medium includes between about 0.2 μM to about 1.0 μM hydrocortisone, such as for example between about 0.3 μM to about 0.7 μM, or between about 0.4 μM to about 0.6 μM hydrocortisone. In yet another example, the adipogenic induction medium includes about 0.5 μM hydrocortisone. In another embodiment, the adipogenic induction medium includes between about 0.2 mM to about 1.0 mM isobutylmethylxanthine, such as for example between about 0.3 mM to about 0.7 mM, or between about 0.4 mM to about 0.6 mM isobutylmethylxanthine. In a particular embodiment, the adipogenic induction medium includes about 0.5 mM isobutylmethylxanthine. In another specific example, the adipogenic induction medium includes between about 30 μM to about 120 μM indomethacine, such as for example between about 40 μM to about 90 μM, or between about 50 μM to about 70 μM indomethacine. In yet another example, the adipogenic induction medium includes about 60 μM indomethacine.

Moreover, adipogenic induction medium can also contain one or more additional additives, such as one or more antibiotics, growth factors, nutrients, or combinations thereof. Generally, adipogenic induction medium includes a minimal essential medium. In one embodiment, the medium is a minimal essential medium, such as α-MEM. The adipogenic induction medium may also be supplemented with serum, such as horse, calf, or fetal bovine serum or combinations thereof. The adipogenic induction medium can have between about 5% by volume to about 25% by volume serum, or about 20% by volume serum, or about 10%. In one embodiment, an adipogenic induction medium is supplemented with 10% FBS and 10% horse serum.

In one, non-limiting example, adipocyte precursor cells can be contacted with an adipogenic induction medium comprising α-MEM, 10% FBS, 10% horse serum, 0.5 μM hydrocortisone, 0.5 mM isobutylmethylxanthine, and 60 μM indomethacine. In a more specific example, the α-MEM is further supplemented with 100 U/ml penicillin and 1 mg/ml streptomycin. Adipocyte differentiation may be expected to occur, for example, in a humidified atmosphere (such as, 100% humidity) of 95% air, 5% CO₂ at 37° C.

The adipocyte precursor cells can be cultured in adipogenic induction medium for a period of time sufficient to increase expression of at least one adipocyte marker. The adipocyte precursor cells can be cultured between about 1 week to about 6 weeks such that adipocyte differentiation may be detected. In other embodiments, adipocyte differentiation may be detected in less than about 1 week, 2 weeks, 3 weeks, 4 weeks, 5 weeks, 6 weeks, and any time period in between.

Adipocyte differentiation may be detected through expression of one or more adipose related markers. As used herein, the term “adipose related marker” includes adipocyte markers, brown adipocyte markers and brown adipose-like markers. The adipose related marker, such as adipocyte markers, may be elevated to a higher level as compared to untreated adipocyte precursor cells. The adipose related marker may be an adipocyte marker or a brown adipocyte marker. Examples of adipocyte markers can include, but are not limited to, fatty acid binding protein 4 (aP2), peroxisome proliferator activated receptor α (PPARα) peroxisome proliferator activated receptor γ (PPARγ), adiponectin (AND or ADIPOQ), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3). Examples of brown adipocyte markers can include, but are not limited to, uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3).

In a particular embodiment, the adipocyte precursor cells are artery-derived cells, such as iMACs, that are cultured in adipogenic induction medium for a period of time and under conditions sufficient to increase expression of at least one adipocyte marker at a higher level as compared to untreated artery-derived cells. In another embodiment, adipocyte precursor cells can also be cultured under conditions sufficient to increase expression of at least one brown adipocyte marker at a higher level as compared to untreated adipocyte precursor cells. The adipocyte marker expression can be increased in treated adipocyte precursor cells at least about 2 fold, 5 fold, 10 fold, 50 fold, 100 fold, 500 fold, 1000 fold, 5000 fold, 10,000 fold, 50,000 fold, 100,000 fold, 150,000 fold, 200,000 fold, 250,000 fold, 300,000 fold, 450,000 fold, 500,000 fold, 550,000 fold, 600,000 fold, 650,000 fold, 700,000 fold, 750,000 fold, 800,000 fold, 850,000 fold, 900,000 fold, or at least about 1,000,000 fold over untreated adipocyte precursor cells. In an exemplary embodiment, the increase in adipocyte marker expression in treated adipocyte precursor cells is at least about 10 fold over untreated adipocyte precursor cells. In another exemplary embodiment, the increase in adipocyte marker expression in treated adipocyte precursor cells is at least about 100 fold over untreated adipocyte precursor cells. In yet another exemplary embodiment, the increase in adipocyte marker expression in treated adipocyte precursor cells is at least about 1000 fold over untreated adipocyte precursor cells.

Differentiation of adipocyte precursor cells, such as artery-derived cells or iMACs, into brown adipose-like cells can be measured by any method known to one of skill in the art. Specific, non-limiting examples are immunohistochemical analysis to detect expression of adipose-related markers using techniques such as Northern blot, RNase protection and RT-PCR. In another embodiment, assays of adipocyte function can be measured, including cytoplasmic accumulation of triglycerides.

For example, fatty acid binding protein 4 (aP2), peroxisome proliferator activated receptor α (PPARα) peroxisome proliferator activated receptor γ (PPARγ), adiponectin (ADN or ADIPOQ), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3)) expression can be measured through assays such as ELISA assay and Western blot analysis. Differentiation of cells can also be measured by assaying the level of mRNA coding for bone-related polypeptides (for example, lipoprotein lipase or peroxisome proliferators-activated receptor γ-2). In an exemplary embodiment, the brown adipose-like cells express at least one adipogenic marker, such as aP2, PPARα, PPARγ, and ADIPOQ. In another exemplary embodiment, the brown adipose-like cells express at least one brown adipocyte marker, such as UCP1, PRDM16, PGC1α, C/EBPβ, CIDEA and ELVOL3. In yet another exemplary embodiment, the brown adipose-like cells express at least one adipogenic marker, such as aP2, PPARα, PPARγ, and ADIPOQ, and at least one brown adipocyte marker, such as UCP1, PRDM16, PGC1α, C/EBPβ, CIDEA and ELVOL3.

Differentiation of adipocyte precursor cells can also be determined by functional potential, such as thermogenic potential or response, of the cells. For example, when oxidative phosphorylation is uncoupled from the ATP synthase channel, proton leak across the mitochondrial membrane generates heat and expends energy. In an exemplary embodiment, proton leak can be measured to determine thermogenic potential of brown adipose-like cells. Additionally, brown adipose-like cells can be activated through exposure to at least one of a catecholamine, like norepinephrine, cyclic AMP and leptin.

Brown adipose-like cells can also be isolated from the differentiation cultures. Cells can be isolated by a variety of methods, including mechanical and/or physical separation methods known in the art.

### Methods of Treatment

In general, the methods and compositions described herein are useful for the treatment of diseases, including metabolic diseases and weight-related disorders. Generally, the methods of obtaining a population of adipocyte precursor cells from a subject, optionally culturing and/or enriching the adipocyte precursor cells to obtain a purified population of adipocyte precursor cells, differentiating the cells as described herein to obtain brown adipose-like cells, and administering the brown adipose-like cells to a subject in need thereof, including a subject that has been diagnosed to be in need of such treatment.

In some embodiments, the methods include identifying a subject in need of treatment (e.g., an overweight or obese subject, e.g., with a body mass index (BMI) of 25-29 or 30 or above or a subject with a weight related disorder) and administering to the subject an effective amount of brown adipose-like cells. A subject in need of treatment with the methods described herein can be selected based on the subject's body weight or body mass index. In some embodiments, the methods include evaluating the subject for one or more of: weight, adipose tissue stores, adipose tissue morphology, insulin levels, insulin metabolism, glucose levels, thermogenic capacity, and cold sensitivity. In some embodiments, subject selection can include assessing the amount or activity of brown adipose tissue in the subject and recording these observations.

The methods and compositions described herein are useful, e.g., for the treatment of metabolic diseases, such as obesity, hyperlipidemia and insulin resistance in a subject, or for treating a disease associated with a lack of mitochondria, e.g., diabetes, cancer, neurodegeneration, and aging.

### Formulations of Brown Adipocyte-Like Cells

In one embodiment, the brown adipose-like cells can be incorporated into pharmaceutical compositions suitable for administration to a subject. A pharmaceutical composition may also include one or more pharmaceutically acceptable carriers. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include water, saline, phosphate buffered saline, dextrose, glycerol, ethanol and combinations thereof. In many cases, pharmaceutical compositions can include isotonic agents, for example, sugars, polyalcohols such as mannitol, sorbitol, or sodium chloride in the composition.

Therapeutic compositions typically must be sterile and stable under the conditions of manufacture and storage. Sterile injectable solutions can be prepared by incorporating the cells in the required amount in an appropriate solution that has been filtered sterilized with one or more ingredients enumerated above.

The compositions of brown adipose-like cells may be administered in a variety of forms. As will be appreciated by the skilled artisan, the route and/or mode of administration will vary depending upon the desired results. The pharmaceutical compositions may include a “therapeutically effective amount” of the brown adipose-like cells. A “therapeutically effective amount” refers to an amount effective, at dosages and for periods of time necessary, to achieve the desired therapeutic result. A therapeutically effective amount of a composition of brown adipose-like cells may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects.

Delivery systems are also provided. In one embodiment, brown adipose-like cells can be part of a kit. The kit can also include additional components, such as one or more pharmaceutically acceptable carriers, as described above. The delivery systems can include reservoirs containing one or more cell types, as described herein, one or more pharmaceutically acceptable carriers, and a delivery device, e.g., a needle or cannula, in fluid contact with the reservoir. In an exemplary embodiment, the brown adipose-like cells can be housed in a single container or chamber of a housing, such as a vial or syringe. A person skilled in the art will appreciate that any housing system known in the art can be used.

Typical exemplary compositions are in the form of injectable or infusible solutions, such as compositions similar to those used for passive immunization of humans. One example mode of administration is parenteral (e.g., intravenous, subcutaneous, intraperitoneal, intramuscular). In one embodiment, the brown adipose-like cells are administered by intravenous infusion or injection. In another embodiment, the brown adipose-like cells are administered by intramuscular or subcutaneous injection. In yet another embodiment, the brown adipose-like cells are delivered to a specific location. In an exemplary embodiment, the brown adipose-like cells can be injected into and delivered to at least one brown adipose tissue depot or any other site with sufficient innervations and vascularity. Local and/or targeted administration of the brown adipose-like cells can be achieved, for example, with a biodegradable matrix. The biodegradable matrix can be an implantable delivery system, wherein the brown adipose-like cells are incorporated into or seeded on the biodegradable matrix.

The brown adipose-like cells can also be delivered with non-resorbable/resorbable scaffolds, such as by encapsulation. Many techniques used for encapsulating cells or preparing scaffolds are known in the art and can be used with the cells disclosed. The encapsulation materials and scaffolds can be made of materials that include, but are not limited to, natural or synthetic polymers, which can be degraded by hydrolysis at a controlled rate and/or reabsorbed.

The brown adipose-like cells can also be included in an implantable device, such as a mesh chamber with a biodegradable core comprising the brown adipose-like cells. The device may be configured to contain and prevent release of cells into the subject's system but allow for exchange of soluble factors. In a particular embodiment, the brown adipose-like cells can be included in the biodegradable core of the implantable device.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals. Brown adipose-like cells can also be incorporated into an extracorporeal device for use as a therapeutic depot that is external to the patient's body.

In one aspect, brown adipose tissue can be increased or augmented through transplantation of brown adipose-like cells. In one embodiment, brown adipose tissue can be increased by about 2%-20% through transplantation of brown adipose-like cells. In another embodiment, brown adipose tissue can be increased by about 5-10% of brown adipose-like cells. In another embodiment, brown adipose tissue can be increased by about 50-100% of brown adipose-like cells. In other embodiments, brown adipose tissue can be increased through transplantation of brown adipose-like cells by at least about 5%, 10%, 20%, 30%, 40%, 50%, 60%, 70%, 80%, 90%, and 100% in either the region/depot of interest or in the patient. The term “transplantation” as used herein refers to the transfer of cells from one body or part of the body to another body or part of the body or from ex vivo to in vivo. The brown adipose-like cells can be autologous, allogeneic, or xenogeneic. If necessary, immune suppression can be administered to prevent rejection of allogeneic or xenogeneic cells. Brown fat like cells can also be encapsulated using a variety of techniques to prevent rejection including encapsulation and other barrier methodologies. An “allogeneic transplantation” or a “heterologous transplantation” is transplantation from one individual to another, wherein the individuals have genes at one or more loci that are not identical in sequence between the two individuals. An allogeneic transplantation can occur between two individuals of the same species, who differ genetically, or between individuals of two different species. An “autologous transplantation” is a transplantation of a tissue or cells from one location to another in the same individual, or transplantation of a tissue or cells from one individual to another, wherein the two individuals are genetically identical.

In one embodiment, adipocyte precursor cells, such as iMACs, MSCs, adipocyte progenitor cells, etc., or brown adipose-like cells can be suspended in a suitable transplant media, such as phosphate buffered saline and other salines. The cell transplant mixture can be injected via a syringe with a needle ranging from 30 to 18 gauge, with the gauge of the needle being dependent upon such factors as the overall viscosity of the adipocyte suspension, into a target location. Needles ranging from 22 to 18 gauge and 30 to 27 gauge can be used.

The term “target site” as used herein refers to a region in the body or a region in a body structure. In some embodiments, the target region can be one or more of the brown adipose tissue depots discussed herein, e.g., a supraclavicular region, the nape of the neck, over the scapula, alongside the spinal cord, near proximal branches of the sympathetic nervous system that terminate in brown adipose tissue depots, around at least one of the kidneys, the renal capsule, the liver, the skin, any other site with sufficient innervations and vascularity or elsewhere.

In addition, target areas are where it is desired to increase or augment brown adispose tissue through administration of brown adipose-like cells, identification of one or more brown adipose tissue depots can be determined on an individualized patient basis by locating brown adipose tissue depots in a patient by imaging or scanning the patient using PET-CT imaging, tomography, thermography, or any other technique, as will be appreciated by a person skilled in the art. Non-radioactive based imaging techniques can be used to measure changes in blood flow associated with brown adipose tissue stimulation within a depot.

In one embodiment, a contrast media containing microbubbles can be used to locate brown adipose tissue. The contrast media can be injected into a patient whose brown adipose tissue has been activated. An energy source such as low frequency ultrasound can be applied to the region of interest to cause destruction of bubbles from the contrast media. The rate of refill of this space can be quantified. Increased rates of refill can be associated with active brown adipose tissue depots.

In another embodiment, a contrast media containing a fluorescent media can be used to locate brown adipose tissue. The contrast media can be injected into a patient whose brown adipose tissue has been activated. A needle based probe can be placed in the region of interest that is capable of counting the amount of fluorescent contrast that passes the probe. Increased counts per unit time correspond to increased blood flow and can be associated with activated brown adipose tissue depots. Because humans can have a relatively small amount of brown adipose tissue and because it can be difficult to predict where brown adipose tissue is most prevalent even near a typical brown adipose tissue depot such as the nape of the neck, imaging a patient to more accurately pinpoint brown adipose tissue depots and can allow more nerves innervating brown adipose tissue to be stimulated with greater precision. Any number of brown adipose tissue depots identified through patient imaging can be marked for future reference using a permanent or temporary marker. As will be appreciated by a person skilled in the art, any type of marker can be used to mark a brown adipose tissue depot, e.g., ink applied on and/or below the epidermis, a dye injection, etc. The marker can be configured to only be visible under special lighting conditions such as an ultraviolet light, e.g., a black light.

### Assessing Treatments

In some embodiments, the methods described herein can include assessing the amount or activity of BAT in the subject following treatment and recording these observations. These post-treatment observations can be compared to the observations made during subject selection. In some embodiments, the subject will have increased BAT levels and/or activity. In some embodiments, the subject will show reduced symptoms. In some embodiments, assessment can include determining the subject's weight or BMI before and/or after treatment, and comparing the subject's weight or BMI before treatment to the weight or BMI after treatment. An indication of success would be observation of a decrease in weight or BMI. In some embodiments, the treatment is administered one or more additional times until a target weight or BMI is achieved. Alternatively, measurements of girth can be used, e.g., waist, chest, hip, thigh, or arm circumference.

These assessments can be used to determine the future course of treatment for the subject. For example, treatment may be continued without change, continued with change (e.g., additional treatment or more aggressive treatment), or treatment can be stopped. In some embodiments, the methods include one or more additional rounds of implantation of brown adipose-like cells to maintain or further reduce symptoms of the metabolic disease in the subject.

### EXAMPLES

One skilled in the art will appreciate further features and advantages can be based on the above-described embodiments. Accordingly, the methods and compositions disclosed are not to be limited by what has been particularly shown and described in the examples or figures, except as indicated by the appended claims. All publications and references cited herein are expressly incorporated herein by reference in their entirety.

### Example 1

### Isolation and Characterization of Brown Adipocyte Precursor Cells

Candidate brown fat progenitor cells were isolated from human internal mammary artery. The ability of these isolated cells (internal mammary artery cells or iMACs) to differentiate to BAT cells was then investigated by exposing the cells to adipogenic differentiation medium.

A portion of the human internal mammary artery was obtained from the National Disease Research Interchange (NDRI, Philadelphia, Pa.). To remove blood and debris, the artery was trimmed and washed in Dulbecco's modified Eagles medium (DMEM-low glucose; Invitrogen, Carlsbad, Calif.) or phosphate buffered saline (PBS; Invitrogen). The entire artery was then transferred to a 50-milliliter conical tube. The tissue was then digested in an enzyme mixture containing 0.25 Units/milliliter collagenase (Serva Electrophoresis, Heidelberg, Germany) and 2.5 Units/milliliter dispase (Roche Diagnostics Corporation, Indianapolis Ind.). The enzyme mixture was then combined with iMAC Growth Medium (Advanced DMEM/F12 (Gibco), L-glutamine (Gibco) penicillin (50 Units/milliliter) and streptomycin (50 ug/mL, Gibco)) containing 10% fetal bovine serum (FBS)). The conical tube containing the tissue, iMAC Growth Medium and digestion enzymes was incubated at 37° C. in an orbital shaker at 225 rpm for 1 hour. The partially digested artery was then transferred to a 50 mL conical tube containing a mixture of fresh enzymes and iMAC Growth Medium and further digested at 37° C. for 1 hour.

The digested artery was then removed from the 50 mL conical tube and discarded. The resulting digest was then centrifuged at 150×g for 5 minutes, the supernatant was aspirated. The pellet was resuspended in 20 milliliters of iMAC Growth Medium. The cell suspensionwas then filtered through a 70-micron nylon BD FALCON Cell strainer (BD Biosciences, San Jose, Calif.). The filtrate was then resuspended in iMAC Growth Medium (total volume 50 milliliters) and centrifuged at 150×g for 5 minutes. The supernatant was aspirated and the cells were resuspended in another 15 milliliters of fresh iMAC Growth Medium and plated into a tissue culture flask that was coated with 50 ug/cm2 bovine type I collagen (Inamed, Freemont, Calif.). The cells were then cultured at 37° C. and 5% CO2.

iMACs were then exposed to adipogenic induction medium (Lonza) for an average period of 3 weeks. Control cells were exposed to adipogenic maintenance medium (Lonza). Either medium was changed every second day. Cells were then fixed for Oil Red O staining and RNA was isolated for quantitative RT-PCR.

### Example 2

### iMACs are Able to Differentiate Down the Adipogenic Lineage as Demonstrated by Oil Red O Staining

Lipid accumulation was analyzed using Oil Red O staining. Cells were fixed with 4% buffered paraformaldehyde solution for 20 minutes at room temperature. Cells were stained for 30 minutes at room temperature with filtered Oil Red O solution (0.5% Oil Red O (Sigma-Aldrich) in isopropyl alcohol). Cells were washed twice with PBS and images were taken with Olympus IX70 microscope camera.

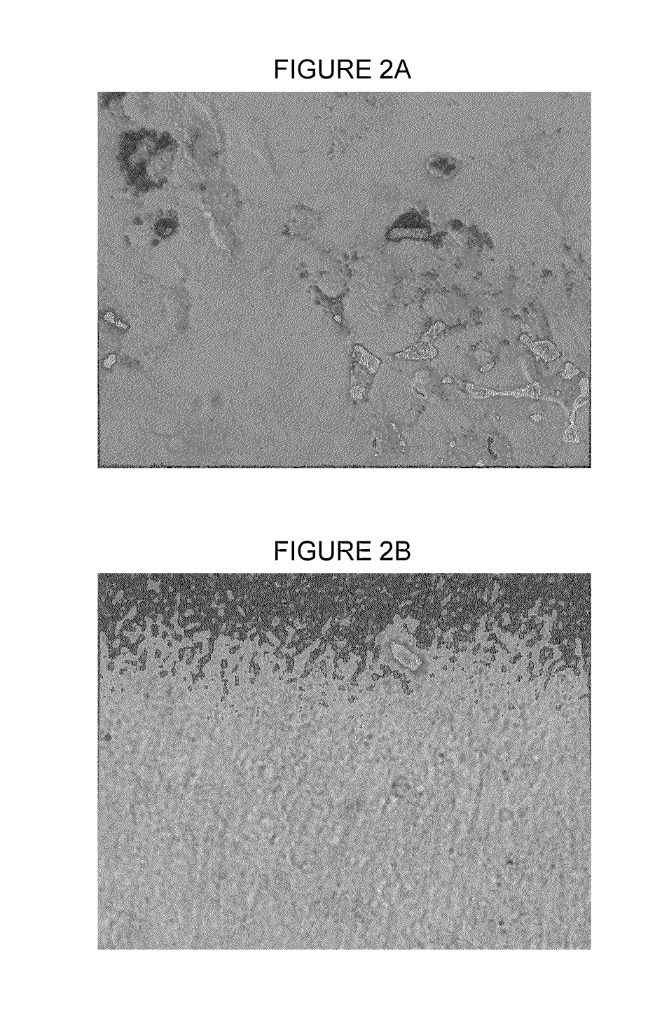

As shown in FIG. 2A, exposure to adipogenic medium caused a marked increase in lipid accumulation in iMACs compared to the control medium (maintenance medium) exposed cell population shown in FIG. 2B, as indicated by the darker appearance of iMACs due to increased Oil Red O accumulation.

This data suggest that iMACs are capable of undergoing adipogenic-lineage differentiation which results in increased lipid accumulation. It further indicated that IMACs are potentially adipose progenitors.

### Example 3

### iMACs Differentiate into BAT-Like Phenotype

iMACs were then exposed to Adipogenic Induction Medium (Adipogenic BulletKit, Lonza) for an average period of 3 weeks. Control cells were exposed to Adipogenic Maintenance Medium (Adipogenic BulletKit, Lonza). Either medium was changed every second day. Before testing, the cells were exposed for 4 hrs to cyclic AMP (cAMP) analogue dibutryl cAMP (Sigma).

Quantitative RT-PCR was then performed to determine the expression levels of both adipogenic and adipogenic markers that are specific to brown adipocytes. The adipogenic markers analyzed were peroxisome proliferator activated receptor gamma (PPARγ), fatty acid binding protein 4 (aP2), and adiponectin (ADN or ADIPOQ). The adipogenic markers specific to brown adipocytes were UCP-1, CIDEA, PPARγ coactivator (PGC)-1α, and ELVOL3.

RNA was isolated according to the manufacturer's specifications (RNeasy Mini Kit; Qiagen, cat #74106). Samples were also treated with DNase I as per kit specification. Final RNA was eluted with 30 μl of ddH20. RNA samples were quantified using 2 μl of each sample and using the NanoDrop 2000 instrument (Thermo Scientific). cDNA was made using the Applied Biosystem “High Capacity cDNA Archival Kit” (Applied Biosystems) using 5 μg of RNA in a final volume of 50 μl (100ng/μl cDNA) according to manufacturer's specifications. PCR was ran by adding 100-200 ng of high capacity cDNA (1-2 μl) plus 7-8 μl Dnase/Rnase-free water plus 10 μl Taqman PCR Master Mix (Applied Biosystems) plus 1 μl desired primer/probe per reaction (20 μl total reaction volume).

TABLE 1
Primer/probes used from Applied Biosystems.

| ABI CAT # | GENE | GENE |
|---|---|---|
| WAT markers | | |
| Hs01086177_m1 | FABP4, | fatty acid binding protein 4, adipocyte |
| Hs00947537_m1 | PPARα | perioxisome proliferator-activated |
| | | receptor alpha |
| Hs01115513_m1 | PPARγ | peroxisome proliferator-activated |
| | | receptor gamma |
| Hs00605917_m1 | ADN or | adiponectin |
| | ADIPOQ | |
| BAT markers | | |
| Hs00154455_m1 | CIDEA | cell death-inducing DFFA-like effector a |
| Hs00222453_m1 | UCP1 | uncoupling protein 1 (mitochondrial, |
| | | proton carrier) |
| Hs00537016_m1 | ELOVL3 | elongation of very long chain fatty acids |
| | | (FEN1/Elo2, SUR4/Elo3, yeast)-like 3 |
| Hs01016719_m1 | PGC1α | peroxisome proliferator-activated receptor |
| | | gamma, coactivator 1 alpha |
| Hs00223161_m1 | PRDM16 | PR domain containing 16 |
| Hs00270923_s1 | C/EBPβ | CCAAT/Enhancer binding protein |
| | | (C/EBP), beta |

PCR was performed in triplicates according to conditions specified by the manufacturer using a 7900 sequence detection system with ABI prism 7900 SDS software (Applied Biosystems, Foster City, Calif.). Thermal cycle conditions were initially 50° C. for 2 min and 95° C. for 10 min followed by 40 cycles of 95° C. for 15 sec and 60° C. for 1 min.

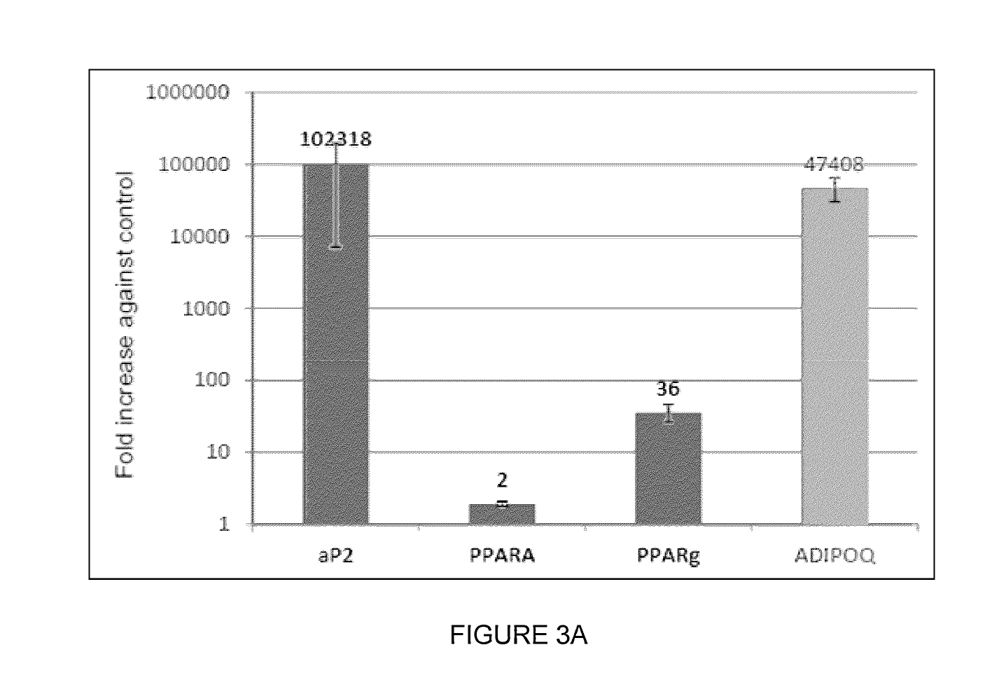

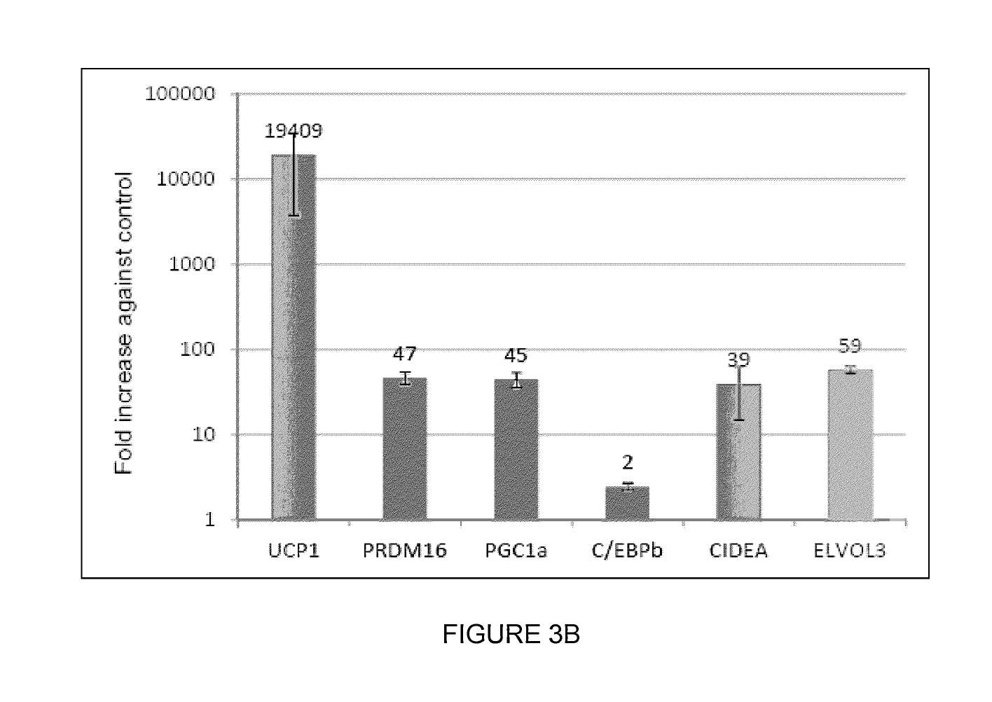

As shown in FIG. 3A and FIG. 3B, a prolonged treatment with adipogenic induction medium resulted in a marked increase in both the adipogenic markers (aP2, PPARα, PPARγ, and ADIPOQ, FIG. 3A) and the brown adipocyte markers (UCP1, PRDM16, PGC1α, C/EBPβ, CIDEA and ELVOL3, FIG. 3B). The lowest induction was observed for PPARα, which presented a 2-fold increase over the control and therefore can be viewed as unchanged. All other markers presented a significant-fold increase over the control. The greatest increase was observed for aP2 and ADIPOQ, which presented a 100,000-fold and an almost 50,000-fold increase over the control, respectively.

These observations suggest that prolonged treatment with adipogenic induction medium promotes the differentiation of iMACs to genuine BAT cells.

To further demonstrate that the iMACs had differentiated to mature brown adipocytes capable of thermogenesis, the adipogenic induction medium-treated cells and untreated control cells were exposed to cyclic AMP (cAMP) analogue dibutryl cAMP (Sigma). This compound mimics the induction of cold-induced thermogenesis and thus triggers the expression of genes involved in thermogenesis, which occurs exclusively in mature brown adipocytes. It is commonly used to functionally characterize brown adipocytes and distinguish brown adipocytes from white adipocytes.

Following treatment, UCP1 expression was analyzed using quantitative RT-PCR, as described above.

As shown in FIG. 3B, iMACs showed approximately a 20,000-fold increase in UCP1 expression following cAMP treatment compared to the control cell population. This observation suggests that iMACs exposed to adipogenic induction medium followed by cAMP treatment differentiate to express brown adipocyte specific markers and have a capacity to respond to catecholamine stimulation to turn on the thermogenic program.

### Example 4

### Ability of iMACs Isolated from Multiple Donors to Differentiate into BAT-Like Phenotype

To investigate functional differences between iMACs isolated from different donors, two additional iMAC lots were isolated, cultured and exposed to adipogenic induction medium as described in Examples 2 and 3. Control conditions involved culture in adipogenic maintenance medium. Levels of the BAT specific markers UCP-1, PRDM16, PGC1α, CIDEA and elvol3 were assessed using RT-PCR.

TABLE 2
Fold induction in the specified brown adipogenic marker genes over
control (maintenance medium). Data shown for two separate
experiments (1) and (2).

| | PGC1α | UCP1 | PRDM16 | C/EBPβ | ELOVL3 | CIDEA |
|---|---|---|---|---|---|---|
| I031011 | 226.2 | 2933.6 | 0.3 | 2.7 | 13.2 | 1259.5 |
| Induction | | | | | | |
| (1) | | | | | | |
| I032511 | 66.0 | 0.3 | 0.3 | 2.3 | 66.5 | 8.0 |
| Induction | | | | | | |
| (1) | | | | | | |
| I031011 | 262.0 | 16.1 | 0.7 | 9.8 | 21.9 | 10791.7 |
| Induction | | | | | | |
| (2) | | | | | | |
| I032511 | 80.5 | 14.2 | 1.2 | 2.6 | 42.3 | 0.5 |
| Induction | | | | | | |
| (2) | | | | | | |

As shown in TABLE 2, iMACs isolated from two additional donors and treated with adipogenic induction medium, resulted in an increase in UCP-1 expression levels as well as other brown adipose tissue markers as compared to the control conditions. These observations suggest that the adipogenic potential of iMACs is not limited to certain genetic background but can be achieved with cells isolated from multiple donors.

### Example 5

### The Effect of iMAC Browning on Mitochondrial Biogenesis

Differentiation of BAT is accompanied by mitochondrial biogenesis, to the extent that the resultant abundant mitochondria and cytochromes cause the brown color of this tissue. The coactivator PGC-1α plays a central role in integrating the transcriptional cascade regulating brown adipogenesis and mitochondrial function PGC-1α stimulates expression of nuclear respiratory factor (NRF)-1 and NRF-2, and coactivates the transcriptional function of these factors on expression of mitochondrial transcription factor A (Tfam), which is a direct regulator of mitochondrial replication and transcription.

TABLE 3
Primer/probes used from Applied Biosystems.

| ABI CAT # | GENE NAME | GENE |
|---|---|---|
| Hs01016719_m1 | PGC1α | peroxisome proliferator-activated |
| | | receptor gamma, coactivator |
| | | 1 alpha |
| Hs00991677_m1 | PGC1β | peroxisome proliferator-activated |
| | | receptor gamma, coactivator |
| | | 1 alpha |
| Hs00192316_m1 | NRF1 | nuclear respiratory factor 1 |
| Hs01082775_m1 | TFAM | transcription factor A, mitochondrial |
| Hs01588974_m1 | CYCS | cytochrome c, somatic |

TABLE 4
Fold induction in the specified brown adipogenic marker genes over
control (maintenance medium). Data shown for two separate
experiments (1) and (2).

| | CYCS | TFAM | NRF1 | PGC1β | PGC1α |
|---|---|---|---|---|---|
| I031011 Induction (1) | 8.2 | 1.7 | 0.6 | 17.6 | 226.2 |
| I032511 Induction (1) | 2.2 | 1.0 | 0.4 | 12.9 | 66.0 |
| I031011 Induction (2) | 18.0 | 2.0 | 0.8 | 33.0 | 262.0 |
| I032511 Induction (2) | 1.1 | 0.3 | 0.1 | 1.6 | 80.5 |

In the iMACs, 3 weeks of treatment with adipogenic induction medium was sufficient to enhance expression of PGC-1α and PGC-1β by 100- to 10-fold, respectively (TABLE 4), accompanied by an approximately 2-18-fold increase in expression of cytochrome C (CYCS). PGC-1α is also known to enhance the transcriptional activity of PPARγ and thyroid hormone receptor on the UCP-1 promoter in brown adipocytes. Thus, the powerful induction of UCP-1 protein expression by adipogenic induction medium in iMACs was likely to be mediated by PGC-1α. There was no change in expression of mitochondrial transcription factor A (TFAM) and nuclear respiratory factor 1 (NRF1).

### Example 6

### More Evidence of BAT Phenotype of iMACs

So far, much of the knowledge on BAT function and development is derived from small mammals where it is well established that BAT is a key metabolic tissue throughout life that helps to maintain body temperature and which regulates energy expenditure and affects body weight. Such studies have clarified the uncoupling process and have started to unravel the cellular precursors and differentiation process of brown adipocytes. In contrast, very little is known about human BAT at the molecular level. Further, based on the research conducted in rodent models, the pharmaceutical industry has developed agonists of the β-3 adrenergic receptor hoping that such compounds would also increase energy expenditure in man. Although agonists of this receptor effectively reduce obesity in rodents, they have failed in clinical trials. Such failures, led many investigators to the conclusion that there are species-specific differences in BAT gene expression. Recently, Svenson et al., have identified genes that are differentially expressed in human BAT and that there are species-specific differences in BAT gene expression. Given these findings, the expression of these genes in iMACs using RT-PCR have been investigated.

RNA isolation and cDNA synthesis was conducted as described in Example 3. The following primers were used to investigate the expression of KCNK3, CKMT1B, COBL, HMGCS2 and TGM2 in iMACs.

TABLE 4
Primer/probes used from Applied Biosystems.

| ABI CAT # | GENE NAME | GENE |
|---|---|---|
| Hs00605529_m1 | KCNK3 | potassium channel, subfamily K, |
| | | member 3 |
| Hs00179727_m1 | CKMT1B | creatine kinase, mitochondrial 1B |
| Hs00391205_m1 | COBL | cordon-bleu homolog (mouse) |
| Hs00194145_m1 | HMGCS2 | 3-hydroxy-3-methylglutaryl-CoA |
| | | synthase 2 (mitochondrial) |
| Hs00190278_m1 | TGM2 | transglutaminase 2 (C polypeptide, |
| | | protein-glutamine- |
| | | gammaglutamyltransferase) |

TABLE 6
Fold induction in the specified brown adipogenic marker genes over
control (maintenance medium). Data shows results of three
replicate experiments.

| | TGM2 | HMGCS2 | COBL | CKMT1B | KCNK3 |
|---|---|---|---|---|---|
| iMAC Maint (1) | 0.1 | 68.2 | 26.6 | 3861.6 | 34.9 |
| iMAC Maint (2) | 0.2 | 199.6 | 21.2 | 660.8 | 81.0 |
| iMAC Maint (3) | 0.1 | 32.9 | 35.1 | 229.0 | 44.0 |

As shown in TABLE 5, all but one (TGM2) of the probed genes were found to be upregulated in the iMACs. This further indicates that iMACs may be bona fide precursors of brown adipocytes and can be easily induced to differentiate along the brown adipose tissue pathway.

### Example 7

### Encapsulation of Brown Fat Like Cells in Natural Material or Synthetic Material

iMACs are differentiated into Brown-fat like cells (BFLC) using the described protocol. Brown fats like cells derived from iMACs or other source cells (white adipocytes, adipocytes progenitors, fibroblast and iPSC), are then aggregated to form spheroid (50-500 μm optimally at 150-200 μm) using methods known in the art (aggrewell plates, low cluster dishes and other). BFLC spheroids are then encapsulated using conventional or conformal or microencapsulation coating methodologies.

BFLC can be microencapsulated in various polymeric hydrogel matrices including alginate, acrylic acid derivatives, polyethylene glycol (PEG) conformal micro-coatings, nanocoatings, cellulose, and/or agarose. Microencapsulated BFLC can be transplanted within the peritoneal space, into existing white adipose depots, omental pouch, subcutaneously or in intramuscular regions.

Another mechanism to house the brown fat cells could be bioartificial implants. In particular, the implants may be thin sheets which enclose cells, may be completely biocompatible over extended periods of time and may not induce fibrosis. The high-density-cell-containing thin sheets can be completely retrievable, and have dimensions allowing maintenance of optimal tissue viability through rapid diffusion of nutrients and oxygen and also allowing rapid changes in response to changing physiology.

### Example 8

### Implantation of Brown-Fat Like Cells Derived from iMACs for Treatment of Obesity

Brown fat-like cells derived from iMACs (or other source cells) can be a good allogeneic source of cells for the treatment of obesity. These BFLC can be used without encapsulation in the presence of immune suppressive agents, or encapsulated or in implantable devices without immunosuppressive agents.

Grafting of BFLC into intramuscular, subcutaneous or into white adipose tissue should be performed in small depots of 100-200 μl suspensions to permit vascularization and innervation of surrounding tissue.

The activity of the implanted BFLC can be modulated by a variety of non-neural stimulation activators including b3-Adrenergic receptor, G-proteins, cAMP, TGR5 bile acids analogs and Type-2 50-deiodinase molecules that activate or up regulate the thermogenic function of the cells.

The efficacy of the brown fat like cells can be assessed in rodent models. Following implantation of BFLC into the animals, BAT activation can be determined through energy expenditure involving continuous measurements of heat output (direct calorimetry) or inhaled/exhaled gas exchange (indirect calorimetry). Indirect calorimetry can be measured through oxygen consumption, carbon dioxide production and/or nitrogen excretion to calculate a ratio that reflects energy expenditure before and after transplantation of BFLC. Thermal imaging methodologies may also be used.

### TERMINOLOGY

All publications, patents and patent applications cited herein are hereby incorporated by reference in their entirety. As used in this specification and the appended claims, the singular forms “a,” “an,” and “the” include plural references unless the content clearly dictates otherwise. The terms used in this disclosure adhere to standard definitions generally accepted by those having ordinary skill in the art. In case any further explanation might be needed, some terms have been further elucidated below.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. The term does not denote a particular age or sex. In a specific embodiment, the subject is human.

As used herein, the term “metabolic disorders” refers to medical conditions characterized by problems with an organism's metabolism. Since a healthy, functioning metabolism is crucial for life, metabolic disorders are treated very seriously. A broad range of conditions including, but not limited to, diabetes (including type 1 and type 2 diabetes), hypo-thyroidism, and obesity are some examples of disorders that can be classified as metabolic disorders. Metabolic disorders can result in excessive weight gain. The term “metabolic syndrome” refers to a cluster of conditions that occur together, and increase the risk for heart disease, stroke and diabetes. Having just one of these conditions such as increased blood pressure, elevated insulin levels, excess body fat around the waist or abnormal cholesterol levels increases the risk of the above mentioned diseases. In combination, the risk for coronary heart disease, stroke and diabetes is even greater. The main features of metabolic syndrome include insulin resistance, hypertension, cholesterol abnormalities, and an increased risk for clotting. Patients are most often overweight or obese.

What is claimed is:

1. Isolated artery-derived, ex vivo differentiated brown adipose-like cells.

2. The brown adipose-like cells of claim 1 further characterized by expression of at least one adipocyte marker selected from fatty acid binding protein 4 (aP2), peroxisome proliferator activated receptor α (PPARα) peroxisome proliferator activated receptor γ (PPARγ), adiponectin (ADN), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3).

3. The brown adipose-like cells of claim 2, wherein the adipocyte marker is a brown adipocyte marker selected from uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3).

4. The brown adipose-like cells of claim 2, wherein the adipogenic marker is expressed in the brown adipose-like cell at higher levels as compared to untreated artery-derived cells.

5. The brown adipose-like cells of claim 1 further characterized by thermogenic potential is stimulated by exposure to at least one of catecholamine and cyclic AMP.

6. The brown adipose-like cells of claim 1, wherein the artery-derived cells are differentiated from internal mammary artery cells.

7. A pharmaceutical composition comprising the brown adipose-like cells of claim 2 and a pharmaceutically acceptable carrier.

8. A method of making brown adipose-like cells comprising:
culturing a population of artery-derived cells in adipogenic induction medium for a period of time and under conditions sufficient to increase expression of an adipocyte marker at a higher level as compared to untreated artery-derived cells.

9. The method of claim 8, wherein the adipocyte marker is selected from fatty acid binding protein 4 (aP2), peroxisome proliferator activated receptor α (PPARα) peroxisome proliferator activated receptor γ (PPARγ), adiponectin (ADN), uncoupling protein 1 (UCP-1), PR domain containing protein 16 (PRDM16), PPAR coactivator-1α (PGC-1α), CCAAT/enhancer binding protein β (C/EBPβ), cell death-inducing DFFA-like effector A (CIDE-A), and elongation of very long chain fatty acids like protein 3 (ELOVL3).

10. The method of claim 8 further comprising isolating the brown adipose-like cells.

11. The method of claim 8, wherein the artery-derived cells are internal mammary artery cells.

12. The method of claim 11, wherein the artery-derived cells are positive for HLA-1 and negative for CD10, CD31, CD34, CD45, CD133, CD141, and KDR/Flk-1.

13. The method of claim 12, wherein the artery-derived cells are additionally positive for CD29, CD44, CD73, CD166, and additionally negative for CD15, CD23, CD24, CD62p, CD80, CD86, CD104, CD117, CD138, CD146, VE-Cadherin, and HLA-2.

14. The method of claim 8, wherein the adipogenic induction medium comprises a compound selected from bone morphogenetic proteins (BMP), peroxisome proliferator-activated receptor gamma (PPARγ), Retinoid X receptor-alpha (RxRα), insulin and T3, a thiazolidinedione (TZD), vitamin A, retinoic acid, insulin, glucocorticoid or agonist thereof, Wingless-type (Wnt), Insulin-like Growth Factor-1 (IGF-1), Epidermal growth factor (EGF), Fibroblast growth factor (FGF), Transforming growth factor (TGF)-α, TGF-β, Tumor necrosis factor alpha (TNFα), Macrophage colony stimulating factor (MCSF), Vascular endothelial growth factor (VEGF) and Platelet-derived growth factor (PDGF).

15. A method of treating a subject comprising:
obtaining a population of artery-derived brown adipose-like cells; and
administering the brown adipose-like cells into a target region in the subject.

16. The method of claim 15, wherein the subject has a metabolic disorder selected from obesity, diabetes or hyperlipidemia.

17. The method of claim 15, wherein the subject is obese and is in need of treatment.

18. The method of claim 15, wherein the artery-derived brown adipose-like cells are autologous to the subject.

19. The method of claim 15, wherein the artery-derived brown adipose-like cells are allogeneic or xenogeneic to the subject.

20. The method of claim 15, wherein the method comprises preparing the brown adipose-like cells as an injectable composition.
